(19) United States
(12) Patent Application Publication
(10) Pub. No.: US 2007/0043434 A1
(43) Pub. Date: Feb. 22, 2007

(54) Biodegradable endovascular stent using stereocomplexation of polymers

(76) Inventors: David Meerkin, Ramat-Beit-Shemesh (IL); Abraham J. Domb, Efrat (IL); Chaim Lotan, Jerusalem (IL)

Correspondence Address: Martin Moynihan;PRTSI, Inc., P.O. Box 16446, Arlington, VA, 22215, US

(21) Appl. No.: 11/206,161

(22) Filed: Aug. 18, 2005

(51) Int. Cl. A61F 2/06

(52) U.S. Cl. 623/1.49

(57) ABSTRACT

A biodegradable stent is comprised of a stereocomplex of polylactide enantiomers. The unique characteristics of stereocomplex polymers allows for the stent to have variable material characteristics along its length, while retaining uniform geometry. Thus, the stent is designed to be flexible upon insertion through the blood vessels while still retaining its rigidity and support upon expansion within the vessel. Furthermore, the material characteristics can be manipulated such that high strengths are possible even with small thicknesses of struts within the stent.

### FIELD AND BACKGROUND OF THE INVENTION

The present invention relates to a biodegradable stent and, more particularly, to a biodegradable endovascular stent using stereocomplexation of polymers.

Restenosis following percutaneous transluminal coronary angioplasty (PTCA) has plagued interventional cardiologists since its inception. The development and application of intracoronary stents has been the first major advance to combat this problem.

Intracoronary stents were initially developed and applied to angioplasty for the treatment of acute closure and dissections. The introduction of this device has allowed interventionists to perform more aggressive angioplasty with larger final lumen diameters without the risk of severe flow limiting dissection and subsequent surgery. These improved immediate post angioplasty results have led to a reduction of restenosis at six months even in non-stented arteries. Furthermore, intracoronary stents have been shown to significantly reduce angiographic restenosis rates in selected coronary lesions compared with balloon angioplasty.

Although intracoronary stents have led to improvements in restenosis prevention, permanent stents may prevent late favourable remodelling of vessels, and furthermore, have been found to cause increased vessel damage and subsequent neointimal formation. This resulting in-stent restenosis has become an even more formidable opponent for the interventional cardiologist. The current challenges are to both prevent and treat in-stent restenosis.

A recent development in preventing in-stent restenosis has been the use of drug coatings. An ideal drug for prevention of restenosis combines inhibition of smooth muscle cell migration and proliferation with local anti-inflammatory effects, and does not develop severe cytotoxicity. The first drug eluting stents contained fibrin or polymer coatings as a reservoir for local drug release. In the 1990s, it was found that some carrier polymers could cause intense inflammatory reactions that would interfere with the antiproliferative effect of the incorporated drugs. However, novel inert polymers, such as a blend of poly(n)-butylmethacrylate and poly-ethylene-vinylacetate as used for the manufacture of the rapamycin-eluting stent, have since been developed and have shown minimal adverse effects.

Although metallic stents are effective in preventing acute vessel occlusion following PTCA and limiting restenosis as described above, particularly when combined with a drug eluting polymer, long term retention of the metallic implant may represent an obstacle to additional treatments, in particular repeat angioplasty and coronary artery bypass surgery. As acute occlusion is limited in general to the first month following implantation, and the restenotic process limited to 6 months, the need for vessel scaffolding is reduced beyond this period. As such, a biodegradable stent offers an alternative that can maintain excellent radial strength for a preprogrammed period to cover the first 9-12 months following deployment, but that following degradation will not inhibit the deployment of another stent or the anastomosis of a bypass graft. It would also allow for improved healing of the vessel in the absence of thrombogenic metallic material. Furthermore, such a device would act as a vehicle for drug delivery allowing for variable rates of degradability with early rapid drug release followed by prolonged maintenance of radial strength.

An example of a prior art stent is one disclosed by Stack et al., in U.S. Pat. No. 5,527,337. The stent, which is porous or has apertures defined therethrough to facilitate tissue ingrowth and encapsulation, is fabricated from polylactic acid in a preferred embodiment. Other suggested polymer materials include certain polyamides, polyanhydrides and polyorthoesters.

Other prior art stents include, for example, a stent disclosed in U.S. Pat. No. 5,551,954 to Buscemi et al., which includes a biodegradable material with a drug releasable at a rate controlled by the degradation rate of the biodegradable material.

Conventional stent design involves a delicate balance between support and flexibility. On the one hand, it is important to have a stent wall which is strong enough to provide the necessary radial support within the vessel. On the other hand, in order to advance the stent to its location within the vessel, the stent must be flexible enough to make its way through the tortuous blood vessels without causing peripheral damage along the way. In order to satisfy both of these requirements, known stent designs include struts having a specific thickness for support, and connectors having smaller thicknesses and consequently low radial strength. The presence of low strength connectors may cause problems including plastic deformation during bending, protrusions into the vessel, or uneven areas which potentially can lead to flow interference. Furthermore, specifically for biodegradable stents, polymer materials used in stent fabrication are generally thicker than metals (typically in the range of about 170 μm) so as to provide sufficient radial strength in the vessel.

There is thus a widely recognized need for, and it would be highly advantageous to have, a biodegradable stent devoid of the above limitations.

### SUMMARY OF THE INVENTION

According to one aspect of the present invention there is provided a biodegradable stent having a body comprised of a stereocomplexed polymer, wherein the body has variable material properties.

According to another aspect of the present invention there is provided a biodegradable stent including a polylactide-based stereo complex, wherein the stereocomplex is configured such that in an unexpanded configuration, the stent is flexible for insertion into a body lumen and in an expanded configuration, the stent is rigid for support of the body lumen.

According to yet another aspect of the present invention there is provided a stent including a wall having a length extending from a proximal end of the stent to a distal end of the stent, the wall having variable material characteristics along the length and a uniform geometric pattern along the length.

According to yet another aspect of the present invention there is provided a stent having a distal end and a proximal end, and a wall extending from the distal end to the proximal end, the wall configured in a tubular shape, wherein the wall is comprised of individual struts arranged in a pattern along the wall, and wherein the struts have a thickness of less than 100 μm.

According to further features in preferred embodiments of the invention described below, the polymer is polylactide and the stereocomplex is formed from L-PLA and D-PLA. According to further features, the stent further includes an elastomer, such as, for example, polyurethane. According to further features, the stent includes a drug. According to yet further features, the body has a uniform geometric pattern.

According to yet further features of the invention, the stereocomplex is configured such that in an unexpanded configuration, the stent is flexible for insertion into a body lumen and in an expanded configuration, the stent is rigid for support of the body lumen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of an interaction between stereoselective polymers;

FIG. 2 is an illustration of an alternating series of crystalline segments and amorphous segments of a polymer structure;

FIG. 3A is a schematic illustration of a stent with defined sections, in accordance with a preferred embodiment of the present invention;

FIG. 3B is an enlarged view of a portion of the stent of FIG. 3A; and

FIGS. 4A-4C are illustrations of several possible embodiments of a stent in accordance with embodiments of the present invention.

### DESCRIPTION OF THE PREFERRED EMBODIMENTS

The present invention is of a biodegradable stent for implantation into a body lumen. Specifically, the present invention provides a stent formed from stereoselective polymers allowing for manipulation of material properties, thus providing versatility in geometric design, strut size and drug delivery options.

Polymer characteristics are affected by various factors, including the type of monomer, the polymer architecture, and molecular weight. In addition, crystallinity of a polymer varies with the stereoregularity of the polymer, such that the higher the stereoregularity, the higher the crystallinity, and the lower the degradation rate.

Stereocomplexation Principles

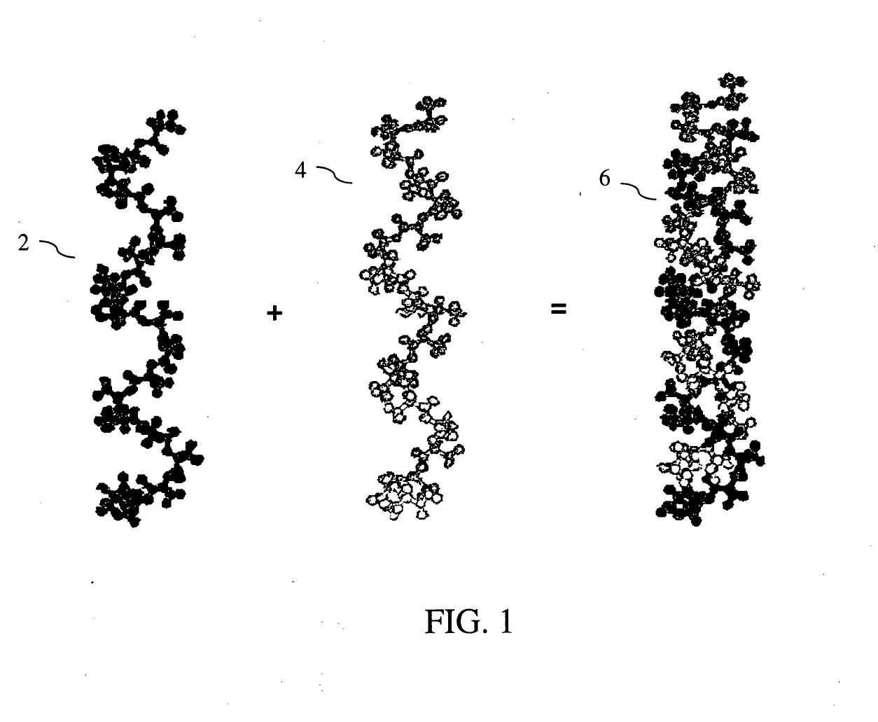

A stereocomplex between polymers is a specific interaction between two chemically identical stereoselective polymers of complementing structures that interlock into a new composite that possesses different physical properties from the individual polymers. An example of this type of interaction is shown schematically in FIG. 1. A first structure 2 and a second complementary structure 4 combine to form a composite 6, having new physical properties due to its altered configuration. As shown in FIG. 1, the stereoselective polymers include chiral segments that have opposite configurations that are able to be combined on a molecular level. The complementing polymers can be either two enantiomeric, optically active, polymer chains with identical chemical composition or two optically active polymers that bear a similar but not identical chemical structure. In the latter case the polymers relate to each other as diastereoisomers. Some polymers can be manipulated to selectively form such configurations, thereby altering the physical properties.

For example, in the case of two polymers, wherein one contains blocks of D-PLA and the other contains L-PLA segments, the D-blocks of one polymer will form a stereocomplex with L-blocks of the second polymer when mixed in solution or in melt. The resulting polymer stereocomplex is insoluble in the solvent that dissolved the original polymers (i.e. acetonitrile) and differs in melting temperature.

In contrast, a mixture of a racemic polymer or a non-chiral polymer will not form a stereocomplex. If for example, racemic D,L-PLA or even enantiomeric L or D-PLA is mixed with polycaprolactone (which is not a stereoselective polymer), no complex will be formed. Neither does racemic PLA or D-PLA form a complex with any (naturally) L-configured polymer, such as polypeptides.

Spinu et al. have reported that D and L enantiomers of poly(lactic acid) form stable stereocomplexes, as reported in “Material Design in Poly(lactic acid) Systems: Block copolymers, Star homo- and copolymers, and Stereocomplexes”, Journal of Macromolecular Science—Pure and Applied Chemistry, 1996; A33:1497-1530.

While stereocomplexes of enantiomeric PLA are known, they usually form a powdery material with limited mechanical properties. The stereocomplexes of the present invention include various copolymers and mixtures that provide enhanced mechanical properties resulting from optimal compositions of segmented stereocomplexation within a polymer composition. For example, block copolymers of D-PLA and DL-PLA with various block lengths may interact with a corresponding block copolymer of L-PLA or L-PLA alone, or with other block copolymers. This interaction can lead to formation of strong and elastic compositions, wherein the strong components are formed by the stereointeraction between complementary blocks along the polymer chains and wherein the amorphous blocks that do not participate in complexation of stereocomplexes contribute to the flexibility and toughness of the composition.

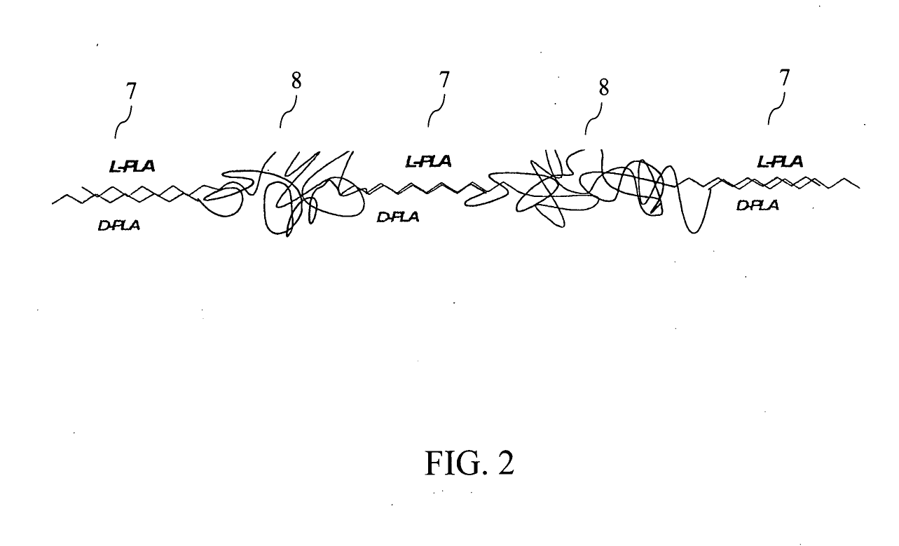

Thus, it is possible to manipulate the packing of the D and L enantiomers, and in this way affect the stereoregularity and consequently the crystallinity of the polymer. This can be done within one structure in a predictable manner, so that the formed structure may have regular and irregular segments depending on the degree of stereocomplexation in each section. An illustration of this concept is shown in FIG. 2. In the construction shown in FIG. 2, an alternating series of relatively crystalline segments 7 and relatively amorphous segments 8 is seen. The crystalline segments are formed by stereocomplexation of equal amounts of L-PLA and D-PLA enantiomers. The amorphous segments are formed by blends that are not stereocomplexed. It should be readily apparent that crystalline segments 7 will have higher strength and rigidity and slower degradation rates than amorphous segments 8. Thus, the material characteristics of the polymer can vary within any given unit in a predictable manner. Furthermore, the crystallinity can be manipulated in a gradual manner so that the degree of crystallinity can vary from amorphous to crystalline, with varying degrees in between. Any pattern of crystalline and amorphous segments can be created, with each configuration having different material properties.

Stereocomplexation can be controlled by polymer composition by method of preparation. With regard to polymer composition, factors which affect degree of stereocomplexation include: the length of the enantiomeric blocks, the presence of non-complexing units within the designated enantiomeric blocks, the properties of the polymer units among the enantiomeric blocks, the degree of polymerization, the structure of the polymer chain (i.e. whether it is linear, branched, star-like or crosslinked), and the composition of interacting polymers, including polymeric chain composition, the relative amount of each polymer, and additives (plasticizers, fillers, stabilizers, etc). For example, triblock copolymers of D-PLA-D,L-PLA and L-PLA are prepared with different block lengths which can be varied from a degree of polymerization of 10 units to 1000 units for each block. The enantiomeric block of D-PLA can be contaminated during its synthesis by adding a minute amount of a contaminant monomer (0.1-10% w/w) including: D,L-lactide. L-lactide, glycolide, caprolactone, trimethylene carbonate and similar monomers. The degree of contamination will determine the complexation efficiency, usually decreasing the complexing interactions. Mixing of polymers of different block compositions such as D-PLA-DL-PLA-D-PLA with multiblock copolymer of L-PLAn-DL-PLAm may provide different results. Thus, polymer composition can be manipulated in order to produce a final stereocomplexed product of varying amounts of crystallinity.

Furthermore, additional polymers or polymer complexes may be selectively combined with the stereocomplexed material to further enhance the material properties of the polymer complex. For example, by combining the properties of polyurethane thermoplastic elastomers (hard segments and soft segments) with the unique stereocomplex concept of D and L lactic acids, strong, flexible biodegradable materials can be obtained. Urethane bonds, for example, have a strong effect on mechanical properties, and may contribute to the elasticity of the device. The urethane bonds may be included in the connections between block chains. For example, blocks of D-PLA, DL-PLA or other chains with hydroxyl end groups can be connected into a long chain by adding a stoichometric amount of a di-isocyanate monomer such as hexamethylene di-isocyanate or isophorone di-isocyanate. Such complexes are likely to demonstrate properties ranging from hard plastics to elastomers.

With regard to the method of preparation, the following factors may affect crystallinity as well: the mixing process, the applied temperature, the molding process, the annealing procedure, solvents used, cooling and heating cycles, pressure and mechanical forces applied, and irradiation. For example, it is possible to alter a molded stent by applying extra heating at certain parts or points so as to focus heating in those areas, thus enhancing stereocomplexation and increasing mechanical properties at particular points.

Stent Design

Several different embodiments of stent design are contemplated in the present invention. It should be readily understood that the designs described herein are merely exemplary, and that any other possible combination of polymers for alteration of material properties and stent design would fall within the scope of the present invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

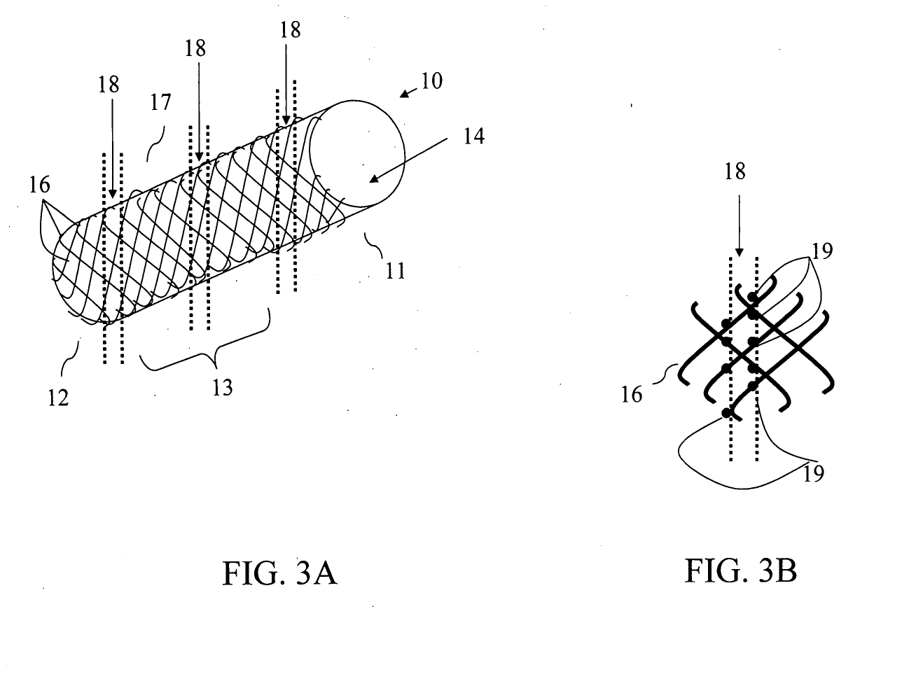

Reference is now made to FIG. 3A, which is a schematic illustration of a stent 10 with defined sections, in accordance with a preferred embodiment. A stent 10 has a distal portion 11, a proximal portion 12, and a middle portion 13, with a lumen 14 therethrough. Stent 10 is comprised of a uniform pattern of struts 16 arranged in a geometric pattern so as to form a wall 17. Designated flexible areas 18 are located along the length of stent 10, so as to allow for flexibility of stent 10. The flexibility is particularly important prior to stent expansion, during delivery of stent 10 to the diseased site. The increased flexibility provides maneuverability within the tortuous system of vessels so as to avoid damage to the vessels along the way.

Reference is now made to FIG. 3B, which is an illustration of an enlarged portion of stent 10, showing the interaction between flexible and non-flexible sections in greater detail. Flexible areas 18 are comprised of a relatively amorphous configuration of, for example, racemic DL-PLA, while the remaining areas are comprised of stereocomplexed combinations of the polymer, resulting in higher rigidity and strength. Flexible areas 18 eliminate the need for separate connectors, as normally found in typical stents. The manufacturing procedure allows for both sections to be present along an individual segment. By forming the stent as one unit, with variable material properties along its length, it is possible to fabricate stent 10 with a seamless joining of flexible and rigid sections. Transition points 19 mark areas of transition between flexible and non-flexible sections. The continuity in the geometric pattern results in a design in which each strut at least partially includes a rigid portion for support. Thus, any lack of rigidity in the flexible areas is compensated for in the rigid sections, which are found along the lengths of the same struts. In addition, all parts of stent 10 may be of the same dimensions (thickness and width), which results in a uniform, smooth design which may help prevent restenosis.

Stent Design Variability

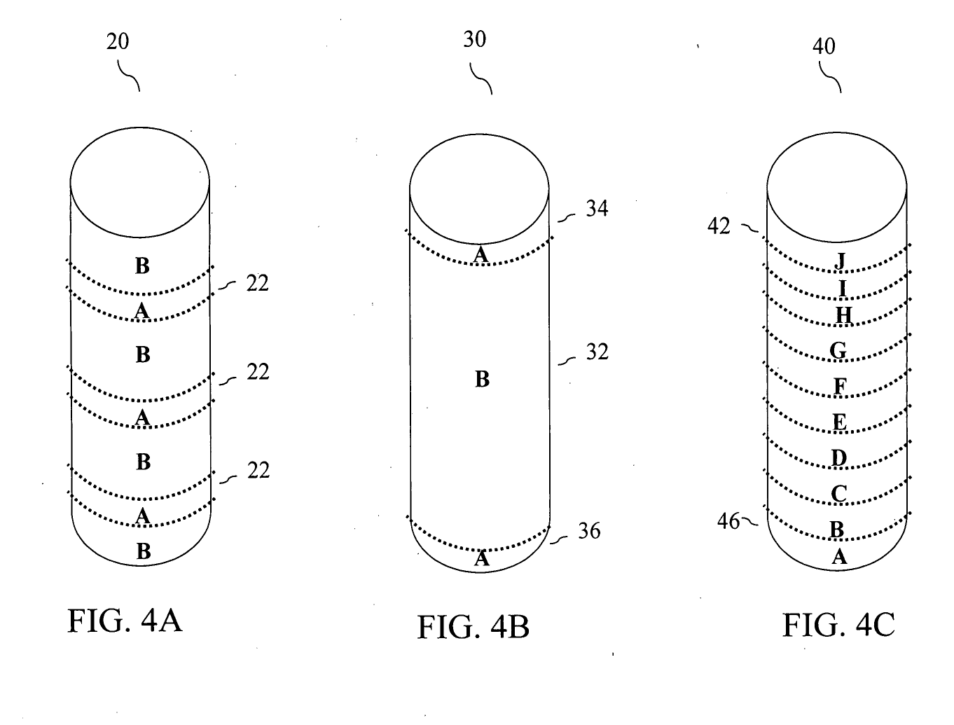

Numerous configurations are possible, using the principles described above to create variable flexibility/rigidity stents. Non-limiting examples of several possible embodiments are illustrated schematically in FIGS. 4A-4C.

In a first embodiment, as shown in FIG. 4A, a stent 20 has a uniformly appearing pattern throughout, with an alternating pattern of high strength portions B and flexible portions A. Transition lines 22 denote transitions between high strength and flexible portions, but it should be noted that these lines are for demonstration purposes only and do not denote an actual line in the stent design. The number and dimensions of flexible portions A may vary. However, in a preferred embodiment, flexible portions A are smaller than high strength portions B, and are utilized so as to provide increased flexibility to stent 20 upon delivery to the site in question. Upon expansion of stent 20 within a vessel, high strength portions B provide support and compensate for any lack of support in flexible portions A.

In a second embodiment, as shown in FIG. 4B, a stent 30 has a mid-portion 32 with high strength portion B, and a distal end 34 and proximal end 36 with softer and more flexible portions A. In this way, upon expansion of stent 30 mid-portion 32 provides the necessary support at the affected site. However, during introduction of stent 10 within the vessel, the edges are not at risk for causing damage to the vessels on its way through. Alternatively, mid-portion 32 has flexibility while distal and proximal ends 34 and 36 have higher strengths.

In a third embodiment, as shown in FIG. 4C, stent 40 has a graded strength, having its lowest strength and highest compliance at a proximal end 46 and progressively getting stronger and less compliant at its distal end 44. Such a design may be useful for tapered vessels, wherein a distal end of the vessel is narrower than a proximal end of the vessel. The use of such a device could enable opening of such a non-uniformly shaped vessel.

Alternative embodiments include one half or one portion of the stent having more rigidity than the other portion, which may be useful for ostial lesions. The transition from rigid to flexible may be abrupt or gradual.

It should be readily apparent that any number of designs may be contemplated, and that the invention is not limited to the examples described herein. The high degree of variability in strength, compliance, elasticity and other material properties enables formation of various types of stents.

Strut Size Variation

Another aspect of stent design which can be improved using a stereocomplex formulation involves strut size. It is desirable to reduce strut size so as to minimize inflammatory reaction and/or restenosis, while still providing the necessary support within the vessel. Efforts have been made to reduce thickness to less than 100 μm, with a more optimal goal being 30-50 μm. By using a stereocomplexed polymer formation such as the one described in the present application, it is possible to produce stronger polymers that can potentially be reduced in size while still providing adequate support to the vessel. If some areas of the stent are too weak to support the vessel at these dimensions (as in the flexible areas described above), the strong sections are designed to compensate and to provide enough rigidity and support for the vessel.

Drug Delivery

A further advantage of the use of stereocomplexed polymers is the ability to provide a suitable carrier for drug delivery. The use of a stereocomplex provides several different possibilities for incorporation of a drug therein, including non-covalent binding of the drug to the polymer complex, or physical entrapment within the complex. Details of the use of stereocomplex polymeric carriers for drug delivery are described more fully in U.S. Pat. No. 6,365,173 to Domb et al., incorporated herein by reference in its entirety. Using the techniques described in Domb, it is possible to create a stent which has a drug encapsulated therein for controlled release upon degradation of the stent in vivo. Furthermore, drugs which are particularly susceptible to early release can be withheld for longer periods of time within the more stable stereocomplex formation.

Polymer Compositions

The polymeric material used for making a stent of the present invention may be comprised of a mixture of several polymers and/or pharmaceutically acceptable fillers including carbon nanotubes, PLA based nano- and micro-spheres prepared by stereocomplexation, hydroxyapatite and other biodegradable additives that can improve the mechanical properties of the polymer composition.

D-PLA-L-PLA stereocomplex nanoparticles are prepared by mixing solutions of D-PLA and L-PLA are mixed at a particular temperature for a specified amount of time (60 degrees Celsius for 5 hours, in accordance with a preferred embodiment) until a milky suspension is obtained. The solids are then separated by centrifugation, resulting in particles in the range of 300-600 nanometers, which are highly porous.

Synthesis of Polymer Structures

Overview:

D-PLA and L-PLA homo- and copolymers of different structures and compositions are synthesized according to accepted methods, including ring-opening and condensation polymerization. Extension of molecular chains or preparation of block copolymers can be achieved by binding the polymer blocks by an amide, ester, carbonate, urethane, urea or other bond, with or without a connecting molecule. The polymers are characterized for their molecular weight by methods such as gel permeation chromatography or, if necessary, by viscosimetry. Besides spectral and thermal analysis, optical rotation can also be measured to confirm their enantiomeric purity.

In one embodiment, polymers are formed by dissolving the appropriate monomers in a solvent. Monomers are dissolved in a suitable solvent, and appropriate polymerization catalysts such as short alcohols, polyethylene glycol (PEG), fatty alcohol or polyalcohol, are added in a particular range, such as 0.1 to 3 mole percent per lactide. The solvent is removed after polymerization is initiated by, for example, solvent evaporation. The molar ratio between the monomer and the catalyst determines the polymer block molecular weight. The lengths and number of blocks are controlled by the number of and amount of each monomer unit added, the sequence of monomer addition to the polymerization mixture at different times, and the amount of catalyst used.

In an alternative embodiment, pre-formed polymer blocks with hydroxyl and carboxylic acid are conjugated via an ester, amide, urethane, phosphate, anhydride, carbonate or other degradable bond. The hydroxyl end groups are reacted with either a diacid chloride (i.e. adipoyl chloride, sebacoyl chloride), alkyl phosphodichloridate, or phosgene to form ester, phosphate or carbonate block conjugates, respectively. Anhydride copolymers are prepared by activating the PLA carboxylic acid end group with acetic anhydride and copolymerized with sebacic acid prepolymer, as described in more detail in Domb et al., J. Poly. Sci. 25:3373 (1987), incorporated herein by reference in its entirety. Multiblock copolymers of PLA are prepared by using a polyalcohol such as pentaerythritol, or glycerol in the catalyst mixture. The structures and block length can be determined by H-NMR and GPC. Typical molecular weight of polymers is in the range of 5,000 to 100,000.

The following examples are various methods of forming block copolymers in accordance with two embodiments of the present invention.

### Embodiment 1

### EXAMPLE 1

### Synthesis of PLA Block-Copolymers

An example of synthesis of polymer structures follows: Homopolymers of PLA are synthesized by dissolving D-lactide or L-lactide in dry toluene at 100° C. and adding a solution of stannous octoate and alcohol as a polymerization catalyst (5% solution in toluene, 0.1 to 3 mole % per lactide). After 3 hours the solvent is evaporated to dryness and the viscous residue is left at 130° C. for an additional 2 hours to yield the polymer. When lactide block copolymers are prepared, the first block, i.e. L-lactide, is prepared in toluene at 100° C. and a second portion of lactide, i.e. D-lactide, is added and polymerization is continued for an additional 2 hours. Following these steps, a third portion of lactide is added and the polymerization is continued. Block copolymers with cyclic hydroxy alkyl acids and cyclic carbonates are prepared in a similar manner, but the second portion is the desired cyclic monomer (caprolactone, trimethylene carbonate, glycolide), instead of lactide.

A tetrablock copolymer including two D-PLA chains and two L-PLA chains is prepared by polymerizing D-lactide with dibenzyl tartarate using stannous octoate as a catalyst. After polymerization at 130° C. as described above, the benzyl protecting groups are removed by hydrolysis or hydrogenation. The free acid groups are esterified with hydroxyl terminated L-PLA in chloroform solution and dicyclohexylcarbodiimide (DCC) as a coupling agent. The lengths of the blocks vary depending on the amount of D-lactide used for polymerization and the L-PLA chain length. This polymer forms an intra and inter-stereocomplexation. Other multiblock polymers include blocks of either or both D-PLA and L-PLA and other biodegradable polymers or poly(oxyalkanes), or include other branching molecules such as mucic acid, pentaerythritol, citric acid and malonic acid 2-methanol.

To form stereocomplexes, the polymers are either dissolved in a solvent or the polymer components are melted together. The polymer mixture is stored under conditions at which the polymers will complex and precipitate out of solution, or cool. The mixture can be formed into a desired shape as it forms, or processed after stereocomplex formation.

Solvents which can be used to dissolve polymers for formation of stereocomplexes include dioxane, chloroform, tetrahydrofuran, ethyl acetate, acetone, N-methylpyrrolidone, ethyl and methyl lactate, ethyl acetate and mixtures of these solvents, and other solvents, such as water, short chain alcohols and carboxylic acids (C5 or below). The particle size of the precipitate is controlled by the selected solvent, the drug and polymer concentrations, and the reaction conditions (temperature, mixing, volume etc.).

As demonstrated by the following examples, a range of copolymers having stereoselective blocks of enantiomorphic PLA may be synthesized and used to form nanoscale structures resulting from specific stereocomplexations. Synthesis of block copolymers having blocks of D-lactide and L-lactide is described. Polymer blocks of molecular weights ranging from 600 to 100,000 daltons are prepared; their molecular weights are estimated by gel permeation chromatography (GPC) and determined by nucleaer magnetic resonance (1H-NMR). L-PLA blocks of 20 to 100 lactide units are conjugated to a biodegradable polyanhydride, polycaprolactone and polyhydroxybutyrate, or to the hydrophilic poly(ethylene glycol) or poly(propylene glycol). The stereocomplexation of these copolymers with short and long chain polymers of D-lactide at different solutions and conditions is characterized by atomic force microscopy (AFM) and related surface characterization methods, such as scanning electron microscopy (SEM) or X-ray photon spectroscopy (XPS). The interaction of poly(D-lactide) and its copolymers with peptides and oligonucleotides to form spontaneous nanoparticles is evaluated as a delivery system to tissues or to cells. Other block copolymers of PLA, such as diblock copolymers of D-PLA-co-L-PLA may also be prepared in the manner stated above, using the proper solvents.

### EXAMPLE 2

### Synthesis of 50:50 wt. % Polylactide/Polycaprolactone

A 250 ml glass flask is charged with 10 g (5.0 mmole) of polycaprolactone diol (PCAP) (Mn=2000), 10 g (69.44 mmole) of L-lactide and dried by azeotrope with 150 ml of Toluene. Toluene is evaporated and the system is stirred for one hour at 150° C. to cause initiation with PCAP diol. Next, 0.35 ml of 0.1M solution of Tin 2-ethylhexanoate (Sn(Oct)₂) in toluene (monomer/catalyst=2000/1) is added. After an additional 2 hours at 150° C. the flask is cooled, and the product polymer is dissolved in a minimal quantity of dichloromethane and precipitated from the mixture of diisopropyl ether:petroleum ether 9:1. The number average molecular weight (Mn) of the related product is about 4000 Da.

Hydroxyl or amino terminated macroinitiators may be used instead of PCAP.

Stereocomplex preparation from those triblock copolymers is carried out according to one of the methods specified above.

Characterization of the copolymers and their stereocomplexes is made by GPC, infrared (IR), differential scanning colorimetry (DSC) and H NMR.

### EXAMPLE 3

### Preparation of Star-Like PLA

Preparation of start-like enantiomeric PLA (D-PLA)₄(DL-PLA)₄₋pentaerithritole is done in one embodiment as follows. A 250 ml round bottom flask is charged with 0.14 gr (0.001 mol) Pentaerythritol (PRT) and 4.32 gr DL-Lactide, and dried by azeotrope with Toluene. After this process, a portion of the Toluene is evaporated and the system is cooled to 120° C. Next, the solution is stirred for a ½ hour at this temperature to cause initiation with the PRT. Next, Sn(Oct)₂ (monomer/catalyst=2000/1) is added, and the reaction is heated to 150° C. and stirred for 2 hours. The reaction is followed with the addition of 4.32 grams of former dried D-Lactide and an additional quantity of Sn(Oct) (additional monomer/catalyst=2000/1). The reaction proceeds for more than 2 hours. The final polymer is a white bulky material having a number average molecular weight of 9400. There are 2 possible ways to prepare stereocomplex gels from this kind of polymers:
i. To prepare (D-PLA)₄(DL-PLA)₄PRT and (L-PLA)₄(DL-PLA)₄PRT and to make stereocomplexes of both of them.
ii. To use one enantiomeric polymer [(D-PLA)₄(DL-PLA)₄PRT for example], and to perform crosslinking by addition of a desired percentage of an opposite homopolymeric enantiomer, such as L-PLA. Using this method, the gel properties may be manipulation by changing the degree of crosslinking and the percentage of the opposite enantiomer. This example demonstrates the use of non-linear enantiomeric polymers for achieving different physical and mechanical properties as the stereocomplex formation is in a three-dimensional shape.

### Embodiment 2

Summary of Preparation Methods:

There are various ways to synthesize multiblock copolymers; the methods proposed below are useful for the preparation of multiblock copolymers with a capability to change a number of parameters together.

Any difunctional molecules may be used in order to gain desired properties, making it possible to get a wide range of properties by changing the composition of the blocks in the copolymer.

Hydrophobic strong and tough melt processable biodegradable fibers and films can be formed. Hydrophobic blocks are used in order to reduce water adsorption. The content of hard segments is increased by using more aromatic blocks or by increasing the percentage of stereocomplexation. These provide a high modulus, which is representative of toughness and strength. In addition, one can control the amount and the size of amorphous blocks; soft segments alter the degradation rate, elongation at break, flexibility, and other relevant properties.

Method 1: Urethane Chain Extension of Triblocks with Hydroxyl Terminals

The method described herein is a two-step synthesis of (ABA)ₙ multiblock copolymers. This method allows the preparation of high molecular weight copolymers with a very broad range of chemical compositions and endless possibilities for modifications. It results in biodegradable thermoplastic poly(ester-urethane). The physicomechanical properties of this type of copolymer can be varied by controlling parameters such as chemical composition, the nature of the B block, and the block length.

The first step is to synthesize the ABA triblock copolymer. Generally, the B block is an OH-terminated oligomer (although it can also be any aliphatic or aromatic diol or diamine), and the A blocks are PLA blocks. The typical procedure for synthesis of ABA triblock copolymer is specified above. By using B blocks with different physicochemical properties we can control the following properties in the target polymer: solubility, stability, percent of water absorption and degradation rate. Examples of the B block are: oligo hydroxybenzoic acid ester diol, polycaprolactone diol, bisphenol A, hydroxy terminated oligo-alanine or glycine. Trimelitic and pyromelitic-amino acid based oligomers may provide excellent rigid segment-B blocks, carboxy-phenoxypropane (CPP) and carboxyphenoxyhexane (CPH) may also be used, but they must be converted to diols or diamine, which can be done by terminating them with propylene oxide, ethylenediamine or ethanolamine.

The A blocks can be semicrystalline PLA blocks produced from L or D-lactide, or amorphous blocks produced from DL-lactide. A block can also be synthesized from any cyclic monomer available for ring opening polymerization, or a combination of some of them.

There is an advantage in using L or D lactic acid for these blocks because it allows for an increase in polymer strength and stability by stereocomplexation between enantiomeric multiblock copolymers.

The second step is to convert the ABA triblocks to high molecular weight (ABA)ₙ multiblock copolymers—poly(ester-urethane) elastomer. The chain extension step, which takes advantage of the “living” nature of OH-initiated polymerization of PLA, can be carried out by using highly reactive difunctional reagents such as different diisocianates, diacyl chlorides, dichlorophosphates, or phosgenes.

Typical Procedure for Preparation of poly(ester-urethane) elastomer:

For this procedure, the triblock copolymers prepared above are used. PLA-PCAP-PLA triblock is melted at 150° C. in a dry flask with Nitrogen atmosphere. An amount of 0.91 g (5.25 mmole) of toluene diisocyanate is added to the flask. After 30 minutes, the flask is cooled to 25° C., and the product polymer is dissolved in 200 ml dichloromethane and recovered by precipitation from hexane. The product polymer is a thermoplastic elastomer having a number average molecular weight of 72,500, a wt. % ratio of A/B of 50/50, a glass transition temperature of −14° C. a melting range of 60-90° C., elongation in break 990%.

The final step in this method is stereocomplexation of polymers having opposite-complementary stereoselective blocks. The stereocomplexation is performed in different ways:
i. Melting together complementary copolymers at different ratios, wherein one is composed of L-PLA block and the other is composed of D-PLA block;
ii. Melting multiblock copolymer with a certain percentage of low molecular weight PLA oligomer that is of opposite stereochemistry to the PLA block of the multiblock copolymer;
iii. Solvent spinning of mixtures of enantiomeric copolymers at various ratios; and
iv. Melt spinning of the enantiomeric copolymers that are melted separately in two chambers, and wherein the hot viscous liquid is united on the fiber line of the melt spinning apparatus, and the stereocomplex is formed in situ.
   Method 2: Chloroformate Chain Extension

The second method which is proposed involves the reaction of α-hydroxy acids oligomers with α,ω-bis(chloroformates). This leads to poly(ester-carbonates) with molecular weights which are at least as high as those of PLA currently obtained by ring-opening polymerization.

The first step is preparation of α-hydroxy acid oligomers by simple polycondensation as specified above. Low molecular weight polymers (and copolymers) are prepared by condensation of lactic acid and other hydroxy acids. The reaction is performed without any catalyst. DL-lactic acid or other difunctional molecules that will improve processability properties of the amorphous blocks are used. For example, polycondensation can be done in the presence of an appropriate amount of hydroxy acid monomer such as ricinoleic acid, hydroxybenzoic acid, and amino acids. The incorporation of ricinoleic acid is advantageous when a lower Tg is desired. Ricinoleic acid is a natural fatty acid with one double bond and a hydroxy group that can successfully ester copolymerized with lactic acid in the same polycondensation procedure.

Ricinoleic acid contributes to the amorphous character of DL-PLA and makes this soft segment more flexible and hydrophobic. The oligomer is a light yellow viscous honey-like liquid. The second step is the condensation of the polyester with α,ω-bis(chloroformates). The general synthetic pathway is summarized in the scheme below.

Bis(chloroformates) of diol oligomers are prepared by simple reaction of the diol with phosgene. Stereocomplexation in this case is performed by the same method described above.

Method 3: Copolymerization by Trans-Esterification

A well-known feature for polyester blends is their exchange reaction, which may occur between reactive functional groups during the melt mixing processes. Alternatively, transesterification can be a useful reaction for the chemical modification of high molecular weight polyesters.

Different catalysts are used in the literature for transesterification. The most abundant are Ti compound catalysts such as, for example titanium (IV) isopropoxide or butoxide and Mg—Al—O-t-Bu.

The main principle of this method is to take high molecular weight PLA, insert segments of different molecules into the sequence by transesterification, and then prepare stereocomplexes from the products.

By using transesterification, two synthetic pathways can be proposed. The first includes transesterification (with an appropriate catalyst) of high molecular weight PLA with any kind of diol, for example a diol that was mentioned above or ethanol amine, and then chain extension by any polyurethane chain extender.

An example of a procedure for transesterification in situ of poly(Lactic acid) (Mn=40,000 Da) is as follows. 5 gr PLA (40,000 Da) are dissolved in 20 ml of dry dichloromethane and placed in a 50 mL round bottomed dry flask equipped with a magnetic stirrer. 1 ml of dry ethanolamine is added. The solution is stirred overnight, and the final product is precipitated in the mixture of diisopropyl ether:petroleum ether 9:1, and dried in a desiccator overnight. The product is characterized by GPC (Mn=3500 Da), IR and NMR.

In order to obtain a high molecular weight polymer after transesterification, 2 gr (0.0006 mol) of the solid is melted in oil bath in 150° C. with stirring under nitrogen. Next, an equivalent quantity of HDI (hexamethylene diisocyanate) is added. The polymerization is continued for 2 hours under nitrogen. The final product is an off-white bulky solid (Mn=70000 Da).

An alternative option uses transesterification difunctional molecules that have a hydroxyl group (—OH) or amino group (—NH₂) on one end, and ethyl or methyl ester on the other end. The R of the difunctional molecule may be any aliphatic or aromatic residue. This pathway enables performance of transesterification followed by polycondensation.

The useful molecule for this reaction includes: p-hydroxy methyl (or ethyl) benzoic ester, 3-(4′-hydroxyphenyl)propionic methyl ester or even ester of N-terminal protected tyrosine. Some hindered molecules may also be used.

An example of a procedure is as follows: 5 gr PLA (40,000 Da) is dissolved in 20 ml of dry dichloromethane and placed in a 50 mL round bottomed dry flask equipped with a magnetic stirrer. 1 ml of dry 3-(4′-hydroxyphenyl)propionic methyl ester is added. The solution is refluxed for 3 hours. The intermediate is characterized by GPC(Mn≅4000 Da).

In order to obtain a high molecular weight polymer after transesterification, the solvent is fully evaporated, and the system is heated to 150° C. with stirring under nitrogen. Then the flask is connected to a vacuum line (0.5 mm Hg) at the same temperature for 1-2 hours (or until the maximal molecular weight is obtained). Every 30 minutes, a sample for GPC is taken. The final product is an off-white bulky solid (Mn=70000 Da).

Stent Formation

Stents are formed by any suitable method, including, for example, injection molding, thick film formation, and jet printing. Injection molding is the process of forcing melted plastic into a mold cavity. Once the plastic has cooled, the part can be ejected. The liquid polymer plastic is often injected under high pressure. The polymer may also be introduced as solid pellets initially, and melted during the process. Using this method, a stent could be formed in parts, wherein different portions of the stent are formed separately. As many individual compartments as desired may be formed, affording the opportunity to use various sections within the same stent, having varying material properties. In thick film formation, stents are formed layer by layer using a template, in which portions of polymer solution may be applied to the template sequentially. Using this method, stents are prepared by first forming a sheet of the desired pattern of the stent and then shaping the sheet into a cylindrical stent by applying an appropriate shaping apparatus using gentle heat with heat welding to form the tube shape. Jet printing is a method which is similar to the method used for printing ink, wherein the ink is substituted by polymers. The polymers may be melted or, alternatively, may be formed as plastic units that are subsequently melted upon application. Different polymer types may be used, each one of which is sprayed onto a predetermined location, thus forming a customized array of polymers with varying material properties. Stents can also be formed by any other suitable engineering methods known in the art.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A biodegradable stent, said stent having a body comprised of a stereocomplexed polymer, wherein said body has variable material properties.

2. The biodegradable stent of claim 1, wherein said polymer is polylactide.

3. The biodegradable stent of claim 1, further comprising an elastomer.

4. The biodegradable stent of claim 3, wherein said elastomer is polyurethane.

5. The biodegradable stent of claim 1, further comprising a drug within said stereocomplex.

6. The biodegradable stent of claim 1, wherein said body has a uniform geometric pattern.

7. The biodegradable stent of claim 1, wherein said stereocomplex is configured such that in an unexpanded configuration, said stent is flexible for insertion into a body lumen and in an expanded configuration, said stent is rigid for support of said body lumen.

8. The biodegradable stent of claim 1, wherein said stent comprises:
a distal end and a proximal end; and
a wall extending from said distal end to said proximal end, said wall configured in a tubular shape, wherein said wall is comprised of individual struts arranged in a pattern along said wall, and wherein said struts have a thickness of less than 100 μm.

9. A biodegradable stent, the stent comprising:
a polylactide-based stereocomplex, wherein said stereocomplex is configured such that in an unexpanded configuration, said stent is flexible for insertion into a body lumen and in an expanded configuration, said stent is rigid for support of said body lumen.

10. The biodegradable stent of claim 9, wherein said stereocomplex is formed from L-PLA and D-PLA.

11. The biodegradable stent of claim 9, further comprising an elastomer.

12. The biodegradable stent of claim 11, wherein said elastomer is polyurethane.

13. The biodegradable stent of claim 9, further comprising a drug within said stereocomplex.

14. The biodegradable stent of claim 9, wherein said body has a uniform geometric pattern.

15. The biodegradable stent of claim 9, wherein said stent comprises:
a distal end and a proximal end; and
a wall extending from said distal end to said proximal end, said wall configured in a tubular shape, wherein said wall is comprised of individual struts arranged in a pattern along said wall, and wherein said struts have a thickness of less than 100 μm.

16. A stent, the stent comprising:
a wall having a length extending from a proximal end of said stent to a distal end of said stent, said wall having variable material characteristics along said length and a uniform geometric pattern along said length.

17. The stent of claim 16, wherein said stent is comprised of a biodegradable polymer.

18. The stent of claim 17, wherein said biodegradable polymer is polylactide.

19. The stent of claim 18, wherein said polylactide is a stereocomplex formation of L-PLA and D-PLA.

20. The stent of claim 18, further comprising an elastomer.

21. The stent of claim 20, wherein said elastomer is polyurethane.

22. The stent of claim 16, further comprising a drug within said stent.

23. The stent of claim 16, wherein said wall is configured such that in an unexpanded configuration, said stent is flexible for insertion into a body lumen and in an expanded configuration, said stent is rigid for support of said body lumen.

24. The stent of claim 16, wherein said wall is configured in a tubular shape, wherein said wall is comprised of individual struts arranged in a pattern along said wall, and wherein said struts have a thickness of less than 100 μm.

25. A stent, the stent comprising:
a distal end and a proximal end; and
a wall extending from said distal end to said proximal end, said wall configured in a tubular shape, wherein said wall is comprised of individual struts arranged in a pattern along said wall, and wherein said struts have a thickness of less than 100 μm.

26. The stent of claim 25, wherein said stent is comprised of a biodegradable polymer.

27. The stent of claim 26, wherein said biodegradable polymer is polylactide.

28. The stent of claim 27, wherein said polylactide is a stereocomplex formation of L-PLA and D-PLA.

29. The stent of claim 27, further comprising an elastomer.

30. The stent of claim 29, wherein said elastomer is polyurethane.

31. The stent of claim 25, further comprising a drug within said stent.

32. The stent of claim 25, wherein said wall is configured such that in an unexpanded configuration, said stent is flexible for insertion into a body lumen and in an expanded configuration, said stent is rigid for support of said body lumen.

33. The stent of claim 25, wherein said wall has a length extending from a proximal end of said stent to a distal end of said stent, said wall having variable material characteristics along said length and a uniform geometric pattern along said length.
